(19) United States
(12) Plant Patent Application Publication
(10) Pub. No.: US 2020/0196500 P1
(43) Pub. Date: Jun. 18, 2020

(54) Kiwi plant named 'AC 497 076'

(50) ;

(71) Applicant: Università degli Studi Udine, Udine (IT)

(72) Inventors: Raffaele Testolin, Udine (IT); Guido Cipriani, Faedis (IT)

(73) Assignee: Università degli Studi Udine, Udine (IT)

(21) Appl. No.: 16/602,868

(22) Filed: Dec. 12, 2019

(30) Foreign Application Priority Data
Dec. 14, 2018 (QZ) 2018/3375

(51) Int. Cl. A01H 6/00

(52) U.S. Cl. PLT/156

(57) ABSTRACT

A new female kiwi plant named ‘AC 497 076’, distinguished by its oblong fruit with brown hairless skin and dark yellow flesh. The fruit is very sweet with low acidity, and matures relatively early.

Genus and species: Actinidia chinensis Planch.

Variety denomination: ‘AC 497 076’.

### BACKGROUND AND SUMMARY OF THE NEW VARIETY

The invention relates to a new variety of kiwi plant (Actinidia chinensis Planch.) named ‘AC 497 076,’ which originated in the course of a plant breeding program at the University of Udine, Italy. Female parent ‘Jintao’ (not patented) was crossed with male parent ‘A134.41’ (not patented), and ‘AC 497 076’ was selected from the resulting seedlings. The new variety has been asexually reproduced by grafting and by in vitro propagation, and has been found to remain true-to-type over successive generations.

‘AC 497 076’ is distinguished from similar variety ‘Soreli’ (U.S. Plant Pat. No. 21,552) by the characteristics set forth in Table 1 below.

TABLE 1
Comparison of ‘AC 497 076’ to ‘Soreli’

| | ‘AC 497 076’ | ‘Soreli’ |
|---|---|---|
| Plant vigor | Medium | Weak |
| Density of hair on | Very sparse | Medium |
| young shoot | | |
| Color of stem shoot | Grey brown | Purple brown |
| on sunny side | | |
| Shape of leaf apex | Emarginate with | Acuminate |
| | cuspidate tip | |
| Leaf basal lobes | Far apart | Slightly |
| | | overlapping |

### DESCRIPTION OF THE PHOTOGRAPHS

FIG. 1 shows a branch, fruits and leaves of ‘AC 497 076’;

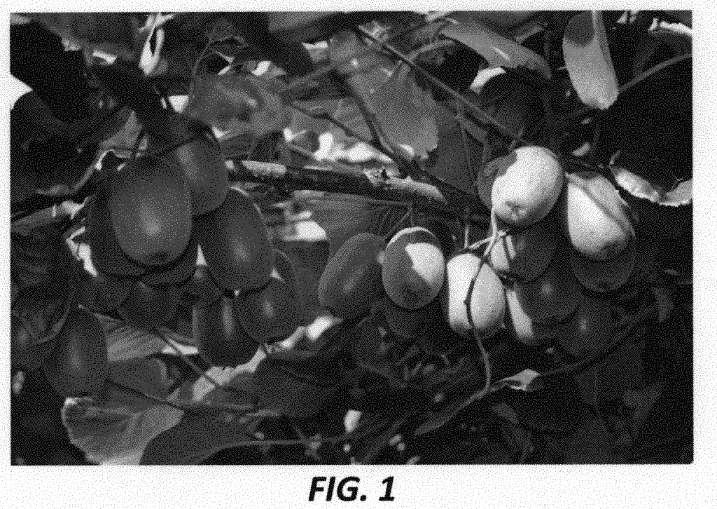

FIG. 2 shows a shoot and young leaves of ‘AC 497 076’;

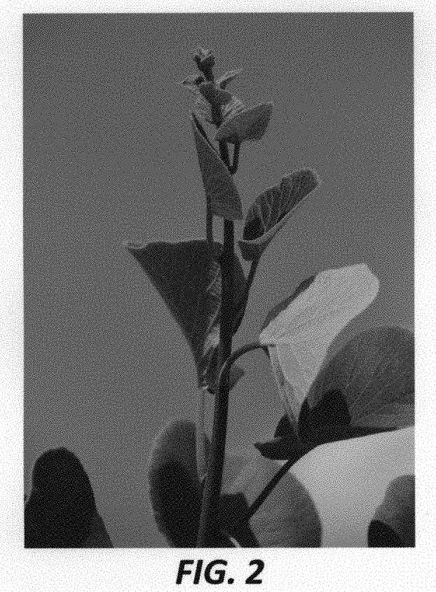

FIG. 3 shows a mature leaf of ‘AC 497 076’; and,

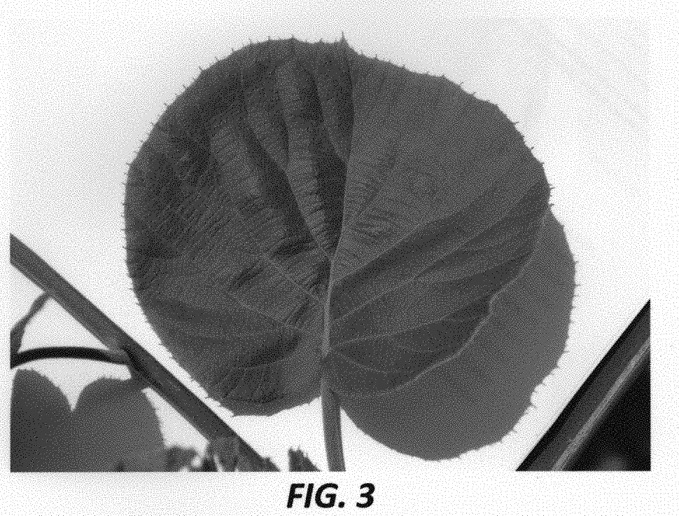

FIG. 4 shows whole and sectioned fruit of ‘AC 497 076’.

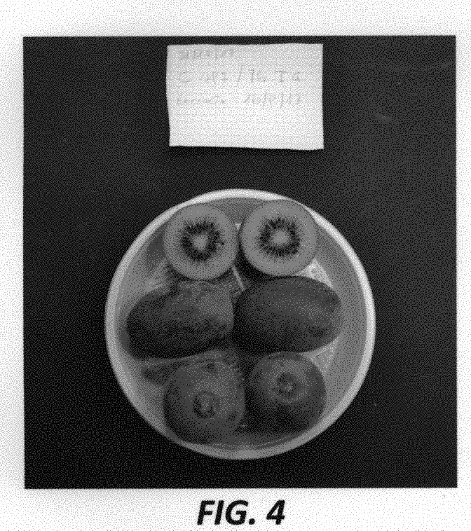

### DETAILED BOTANICAL DESCRIPTION

The characteristics described herein will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Plant sex: Female.
Self-fruit setting: Absent.
Plant vigor: Medium.
Density of hairs on young shoot: Very sparse.
Anthocyanin coloration of growing tip on young shoot: Absent or very weak.
Stem:
   Thickness.—Medium.
   Color of shoot on sunny side.—Grey brown.
   Texture of bark.—Smooth.
   Density of hairs.—Absent or sparse.
   Size of lenticels.—Small.
   Number of lenticels.—Few.
   Prominence of bud support.—Weak.
   Presence of bud cover.—Absent.
   Leaf scar.—Moderately depressed.
   Pith.—Lamellate.
Leaf:
   Shape.—Obovate.
   Length/width ratio.—Moderately compressed.
   Shape of apex.—Emarginate with cuspidate tip.
   Basal lobes.—Far apart.
   Number of ciliate serrations.—Many.
   Density of hairs on upper side.—Medium.
   Density of hairs on lower side.—Absent or very sparse.
   Intensity of green color of upper side.—Light.
   Color of lower side.—Light green.
   Variegation.—Absent.
   Length of petiole relative to blade.—Large.
   Anthocyanin coloration of upper side.—Medium.
Flowers:
   Inflorescence type.—Dichasium.
   Number of flowers.—Medium.
   Flower bud.—Position of first spike Low.
   Number of sepals.—Medium.
   Main color of sepals.—Green.
   Density of sepal hairs.—Absent or sparse.
   Diameter.—Medium.
   Arrangement of petals.—Overlapping.
   Shape in profile.—Convex.
   Number of styles.—Medium.
   Attitude of styles.—Semi-erect.
   Petal.—Color on adaxial side Yellowish white.
   Petal.—Second color on adaxial side Red spot on one petal.
   Anther color.—Yellow orange.
   Pollenizers.—Belen, Zuva, Matua (not patented).
Fruit:
   Weight.—High.
   Length.—Medium.
   Width.—Medium.
   Length/width ratio.—Medium.
   Shape.—Oblong.
   Shape in cross section at median.—Circular.
   Stylar end.—Strongly depressed.
   Presence of calyx ring.—Medium.
   Shape of shoulder at stalk end.—Weakly sloping.
   Length of stalk.—Medium.
   Length of stalk relative to length of fruit.—Medium.
   Conspicuousness of lenticels on skin.—Weak.
   Hairiness of skin.—Absent.
   Color of skin.—Medium brown.
   Adherence of skin to flesh.—Strong.
   Color of outer pericarp.—Dark yellow.
   Color of locules.—Medium yellow.
   Width of core relative to fruit.—Medium.
   General shape of core in cross section.—Circular.
   Color of core.—Yellow white.
   Sweetness.—High.
   Acidity.—Low.
Timing:
   Time of vegetative bud burst.—Early.
   Time of beginning of flowering.—Medium.
   Time of maturity for harvest.—Early.

We claim:

1. A new and distinct kiwi plant substantially as described and illustrated herein.
